(19) United States
(12) Patent Application Publication
(10) Pub. No.: US 2005/0119521 A1
(43) Pub. Date: Jun. 2, 2005

(54) Vacuum exercise device for promoting expansion of soft-tissue

(76) Inventor: Rex Lewis Pitcher, Preston, ID (US)

Correspondence Address: PATE PIERCE & BAIRD, 215 SOUTH STATE STREET, SUITE 550, PARKSIDE TOWER, SALT LAKE CITY, UT, 84111, US

(21) Appl. No.: 10/725,619

(22) Filed: Dec. 2, 2003

(51) Int. Cl. A61F 5/00

(52) U.S. Cl. 600/38

(57) ABSTRACT

An apparatus for promoting radial and axial expansion of soft tissue, the apparatus comprising: (1) a chamber having a wall defining an interior thereof and supporting an ambient pressure therein; (2) a vacuum source connected to the chamber to reduce the ambient pressure therein; and (3) a membrane sealingly connected to the chamber and characterized by a constant of elasticity selected to provide a bias resisting the reduction of ambient pressure against a soft tissue member in the membrane in order to reduce localized trauma to the soft tissue due to the reduction of ambient pressure. A method for exercising a member formed of soft tissue, the method comprising: (1) providing a chamber having a wall of fixed dimension; (2) providing an evacuation device to reduce ambient pressure in the chamber; (3) providing a membrane, substantially tubular in shape, substantially elastic, having an interior passage, having a first end sealing securable to the wall of the chamber, and having a second end extendable into the chamber a length greater than the length of a member comprising soft tissue; (4) occluding the interior passage of the membrane by the member, in a flaccid state; (5) operating the evacuation device to reducing the ambient pressure in the chamber; (6) drawing the member into the interior passage by virtue of a pressure differential between bodily vascular pressure and the ambient pressure; and (7) expanding the member axially and radially due to the pressure differential.

### BACKGROUND

1. Field of the Invention

This invention relates to apparatus and methods for promoting the expansion of soft tissue and more particularly devices providing a reduced pressure environment.

2. The Background Art

Often it may be desirable to expand specific areas of soft tissue on a human body. Reasons for expansion may simply relate to cosmetic enhancement or may be related to the alleviation of a specific dysfunction, for example erectile dysfunction (ED). Specific areas for soft tissue expansion may include various appendages or members, including the female breast and the male penis. Numerous apparatus and methods are available to promote soft tissue enlargement and enhancement. Therapeutic apparatus and methods have largely been directed toward: (1) surgery, (2) chemical compositions, and (3) mechanical devices. Chemical composition therapy for ED has recently received much attention with the introduction of a new pharmacological class.

Additional therapeutic measures for ED have been directed to psychological support, alternative sexual techniques, and lifestyle modifications. Psychological support, alternative sexual techniques and lifestyle modifications target relatively specific and relatively small subsets of individuals who experience ED.

Surgical treatment for ED may include implantation of a penile prosthesis or penile revascularization. These surgical techniques may be subject to malfunction, perforation and may additionally present a risk of infection. Surgical therapy for enlargement of breast tissue typically involves the surgical implantation of prosthetic materials. These surgical options for soft tissue enhancement and enlargement are not easily undertaken and have significant risk and cost.

Pharmacological management may be systemic or local depending on the agent and the route of administration. Systemic agents include alpha-adrenergic antagonists (e.g., yohimbine, phentolamine), domainergic agonists (e.g., apomorphine) and phosphodiesterase inhibitors (e.g., sildenafil). Systemic agents can have significant untoward or side effects and may also have substantial cost. These agents are typically administered orally or sublingually.

Local pharmacological agents include intracorporal injection of prostaglandin (e.g., CAVERJECT) and intraurethral administration of prostaglandin (e.g., MUSE). These agents can be costly and require an injection or administration as a urethral suppository and thus have not been widely favored over other ED therapies.

Mechanical device therapy for ED includes constriction rings and vacuum (e.g., environmental pressure reduction) devices. Constriction rings, sometimes referred to as constriction clamps, constriction bands, restriction clamps, restriction rings or restriction bands, are used to prevent early detumescence or to retain a vacuum generated erection, upon removal of a vacuum source. Vacuum device prior art has focused primarily in three directions: (1) breast tissue enhancement apparatus, (2) closed-chamber systems, including pump apparatus, and (3) retention clamp apparatus. These directions depend on the alteration of the pressure atmosphere surrounding the soft-tissue.

Pressure is relative. If vascular flow or pressure is insufficient, environmental pressure may be reduced to provide a relative pressure difference. In general practice, an area of soft tissue where enlargement is desired is placed into a chamber and sealed such that a separate atmosphere can be maintained around the soft tissue. Utilizing a vacuum source, typically a vacuum tube or chamber and a pump mechanism, air is evacuated from a vacuum chamber. The resulting alteration in pressure may lead to tumescence of the soft tissue with blood, filling with other fluids, or both. Thus, vacuum devices are commonly combined with a constricting device, typically in the form of an elastic or otherwise flexible clamp to maintain the soft-tissue enlargement for a short-term duration.

As appreciated by those skilled in the art, the current state of vacuum devices leaves a number of disadvantages. Vacuum devices are commonly intended for short-term soft-tissue enlargement, typically to facilitate sexual intercourse. Short-term, vacuum device soft-tissue enlargement for sexual intercourse requires the use of a constricting device to retain blood in the soft tissue and maintain enlargement.

Vacuum enhancement devices subject soft tissue to significant localized damage. This damage may result from device (e.g., chamber) pressure on tissues, localized tissue weakness under vacuum, local vascular weakness, or other uneven responses to application of negative pressures (i.e., reduced pressure atmosphere) generated by a vacuum or suction device. Damage may be manifest in the form of pain and discomfort, bruising, discoloration, or function, such as ejaculation impairment. Also, vacuum devices can be costly and less effective than desired.

A safer and more commercially practicable apparatus and method for promoting both temporary and permanent expansion of soft tissue, in multiple dimensions, without causing soft tissue damage are therefore needed. Such apparatus and methods may be directed toward the development of a system supporting an exercise conditioning program.

### OBJECTS AND BRIEF SUMMARY OF THE INVENTION

One expected benefit of an apparatus and method in accordance with the present invention is to provide apparatus for promoting radial and axial expansion of soft tissue in a human. An apparatus comprising a chamber having a wall defining an interior capable of supporting a reduced pressure environment may be evacuated by a vacuum source connected to the chamber to reduce the ambient pressure, or a membrane may be sealing connected to the chamber, characterized by a coefficient of elasticity selected to provide a bias pressure against a bodily member in the chamber.

Another benefit resulting from certain embodiments of the present invention is to provide a method for promoting permanent soft tissue expansion in a soft tissue member, such as by providing an apparatus for soft tissue expansion, deploying an apparatus for soft tissue expansion and conducting a soft tissue expansion exercise.

Another embodiment may provide an apparatus for promoting soft tissue expansion in a human. The apparatus may include a vacuum chamber, a vacuum pump, a vacuum tube, a bushing, a membrane and a sealant, such as a gel. Such a system may provide a means for promoting the expansion of soft tissue, such as erectile tissue of male genitalia. An apparatus and method in accordance with the invention may provide a system for promoting the expansion of soft tissue as well as vascular enlargement, flow ease, and flexibility with permanent effect.

Certain embodiments of an apparatus and method in accordance with the invention may provide a system for more even distribution of axial and radial stresses along soft tissue, such as a bushing and a membrane for controlling contact pressure against tissues. A bushing may be in a cylindrical shape, having an afferent (inlet) aperture, an efferent (exit) aperture, and a lip. A membrane may include a cylindrical neck, a tapered base, a rolled flange, an afferent aperture, and an efferent aperture.

Additionally, it is a feature of certain embodiments of an apparatus and methods in accordance with the present invention to provide apparatus for minimizing soft tissue functional damage, such as bruising, tissue rupture, and impairment of ejaculation, by use of a membrane with a coefficient of elasticity selected to equalize local pressure and deflection in a way to minimize trauma to soft tissue.

It is an additional feature of certain apparatus and methods in accordance with the present invention to provide apparatus and methods for minimizing soft tissue trauma, such as bruising, discoloration, deformity, pain and tenderness, (e.g., such as may result from blood vessel aneurysm, microaneurysm, or disruption during a soft tissue expansion exercise) by use of a sealant, such as a gel, applied to a membrane for an even distribution of radial and axial stress along soft tissue.

It is another feature of certain embodiments of systems and methods in accordance with the present invention to minimize soft tissue trauma during a soft tissue expansion exercise by use of an extended bushing lip providing additional support against an abdominal wall. Additionally, they provide a method for soft tissue expansion by providing a vacuum source, vacuum chamber, bushing, and membrane, followed by fitting, application, insertion, and evacuation, followed by reflood.

Additionally, an apparatus and method in accordance with the present invention provide for promoting an erection in a human penis by alteration of a pressure balance, pressure transduction, an application of radial and axial forces, an increased blood flow, and a vascular expansion.

The present invention relates to apparatus and methods for promoting the expansion of soft tissue in a human. An apparatus promoting the expansion of soft tissue may employ the use of a vacuum source, a support bushing and a membrane. A vacuum source may further employ the use of a vacuum chamber, vacuum tubing and a device for evacuating air from the vacuum chamber. A support bushing may be used to provide a stable base for resting a vacuum chamber. A bushing lip may extend outwardly from the center of a bushing aperture and may serve to support a vacuum chamber wall. In an alternative embodiment of the present invention, a bushing lip may extend outwardly to also serve as an abdominal wall support.

An abdominal wall support may lessen physical trauma to soft tissue which may be possible during soft-tissue exercise and may limit drawing of abdominal tissue into the vacuum chamber. A membrane may be used to provide a greater vacuum seal against soft tissue. A membrane may be threaded through a support bushing to provide a system for achieving uniformity of the section modulus of a columnar member and for a uniform distribution of radial and axial forces along soft tissue.

### BRIEF DESCRIPTION OF THE DRAWINGS

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a process flow diagram illustrating a method, through exercise, for promoting vascular health and the permanent expansion of soft tissue in a human using an embodiment of an apparatus in accordance with the present invention comprising a soft-tissue expansion apparatus, deployed for soft-tissue expansion exercises;

FIG. 2 is a process flow diagram illustrating a process providing a soft-tissue expansion apparatus of the method of FIG. 1 in one embodiment comprising providing a vacuum source, providing a vacuum chamber, providing a bushing, and providing a membrane;

FIG. 3 is a process flow diagram illustrating deploying a soft-tissue expansion apparatus as in FIG. 1 in an embodiment comprising fitting, application, and insertion;

FIG. 4 is a process flow diagram illustrating one embodiment of conducting a soft-tissue expansion exercise in accordance with FIG. 1 with an embodiment comprising evacuation and reflood;

FIG. 5 is a process flow diagram illustrating a method for exercises promoting an erection in a human penis using an embodiment in accordance with the present invention, comprising alteration of pressure balance, pressure transduction to a membrane, pressure distribution, application of radial and axial force, increased blood flow, and vascular expansion;

FIG. 6 is a side, cross-sectional view of one embodiment of an apparatus in accordance with the present invention, illustrating an apparatus for promoting soft tissue expansion in a human;

FIG. 7 is a side, perspective view of a membrane used for providing a more even distribution of axial and radial forces, deflection, and localized stresses along soft tissue in one embodiment of an apparatus in accordance with the present invention;

FIG. 8 is a side, perspective view of a bushing useful for securing the membrane of FIG. 7 and for providing a more even distribution of axial and radial forces, deflections, and stresses along soft tissue in an embodiment of an apparatus in accordance with the present invention;

FIG. 9 is a side, perspective view of an alternative embodiment of a bushing useful for securing the membrane of FIG. 7 and for providing a more even distribution of axial and radial forces, deflections, and stresses along soft tissue;

FIG. 10 is a side, perspective view of one embodiment of the initial assembly of a system providing an even distribution of axial and radial forces and stresses along soft tissue, comprising a bushing and a membrane;

FIG. 11 is a side, perspective view of a final assembly for providing a more even distribution of axial and radial stresses and deflections along soft tissue, comprising a bushing and a membrane;

FIG. 12 is a side, cross-sectional view of a membrane used for providing a more even distribution or dispersion of axial and radial stresses and deflections along flaccid soft tissue including a physiological illustration of the method of FIG. 5;

FIG. 13 is a side, cross-sectional view of a membrane used for more even distribution of axial and radial stresses and deflections along erect soft tissue in one embodiment, a physiological illustration of the method of FIG. 5.

FIG. 14 is a side, cross-sectional view of a one embodiment of an apparatus and method in accordance with the present invention in which one or more restriction bands are positioned on the outer diameter of a vacuum chamber.

FIG. 15 is a side, cross-sectional view of one embodiment of an apparatus and method in accordance with the present invention in which one or more restriction bands are positioned on the base of the penis to prevent early detumescence of an erection or to retain rigidity of a vacuum generated erection, upon removal of a vacuum source, for a period of time.

### DESCRIPTIONS OF THE PREFERRED EMBODIMENTS

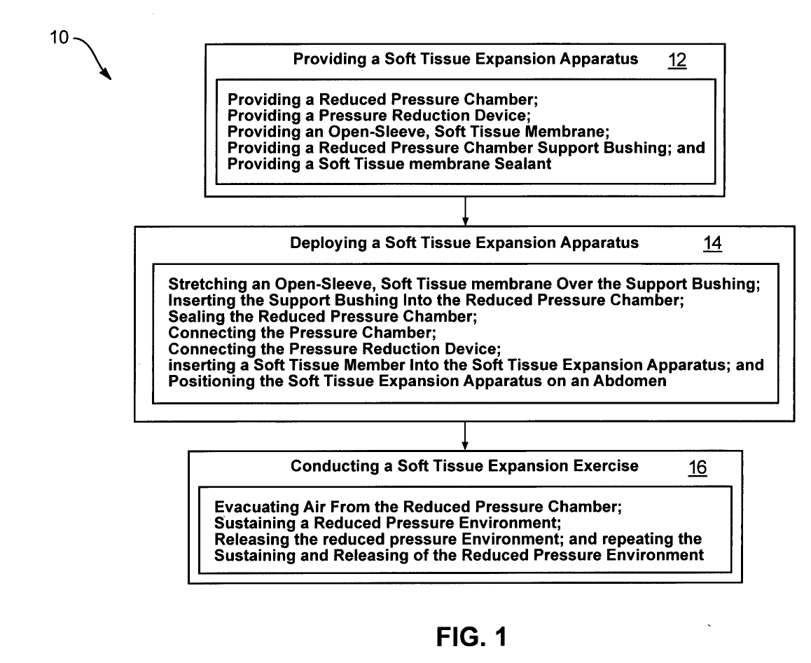

It will be readily understood that the components of the present invention, as generally described and illustrated in the Figures herein, could be arranged and designed in a wide variety of different configurations. Thus, the following more detailed description of the embodiments of the system and method of the present invention, as represented in FIGS. 1 through 13, is not intended to limit the scope of the invention, as claimed, but is merely representative of certain embodiments contemplated for implementing the invention.

Likewise, the steps of the methods disclosed herein are illustrative and are not intended to limit the steps performed or the ordering of steps performed. The presently disclosed embodiments implemented in accordance with the invention will be best understood by reference to the drawings, wherein like parts are designated by like numerals throughout.

An erection is a physiological event wherein a body part or organ is enlarged due to erectile tissue becoming filled or distended with blood. This filling or distension tends to tighten the erectile tissue. Erection is commonly used to describe the blood distension of erectile tissue within certain tissues in both male and female genitalia and female nipples in mammals. Erectile dysfunction, commonly referred to as “impotence”, can be defined as the inability to achieve or maintain an erection or the achievement of an erection which is not satisfactory for conducting sexual intercourse. The extent of swelling or enlargement is sometimes described as tumescence. An unsatisfactory erection may include inability to achieve optimal tumescence or the too early occurrence of detumescence.

Erectile dysfunction has a prevalence in the United States of approximately 10-20 million individuals. Approximately 40% of those over age 60, and approximately 57% of those over age 70 experience ED. Multiple causes can lead to ED, including endocrine disorders, vascular causes, neurological conditions, side effects of medications, substance abuse disorders. In one of five (20%) of the cases, the cause is psychogenic. In those individuals with spinal cord injury, approximately 50% may develop ED. These statistical data suggest there are sufficient numbers of individuals who suffer from ED that it would be desirable to develop apparatus and methods for the expansion of soft tissue.

Any number of suitable apparatus and methods may be utilized for expansion of soft tissue. These known apparatus and methods are often associated with significant trauma to soft tissue and are typically directed to a transient expansion of soft tissue for the purpose of achieving an erection for sexual intercourse. The present invention is directed to apparatus and methods for promoting both temporary and permanent soft tissue expansion such as through utilization of one or more of a partial vacuum, pressure reduction, or suction device, bushing, membrane, and a sealant in combination with an exercise conditioning program.

A common example of soft tissue enhancement for a short-term event occurs in the case of initiating and maintaining an erection in a male for sexual intercourse. Stated another way, a vacuum enhancement apparatus changes ambient pressure around a flaccid member causing enlargement, often described as an erect member. In this event, a vacuum enhancement device is placed over a flaccid male member and the air is evacuated to result in an erect member. Upon removal of the vacuum enhancement device, the pressure change around an erect member will soon equalize with ambient atmosphere and the erection will rapidly dissipate back to a flaccid state.

To overcome this disadvantage, vacuum enlargement apparatus are often combined with the use of a constricting device. A constricting device, typically in the form of an elastic or otherwise flexible clamp, in an open (e.g., extended, stretched, large diameter) position, is placed on the base of a flaccid member prior to placement of a vacuum chamber. These constricting devices are sometimes referred to as clamps, rings or bands. Following, around, or near generation of a vacuum and a resulting conversion from a flaccid to an erect state, a clamp is closed. The clamp closes prior to, or immediately after removal of the vacuum chamber to promote maintenance of the erect member for an extended period of time.

Certain embodiments of an apparatus and method in accordance with the invention are directed toward the establishment of an exercise conditioning program. Through repetitive use of these embodiments over time, an exercising conditioning program promotes improved vascular conditioning and flexibility, better flow, an enlarged and healthier soft tissue, and greater optimum erection tumescence.

Studies conducted by the inventors indicate apparatus and methods for promoting the expansion of soft tissue may result in healthier soft tissue. In addition, in at least one embodiment of an apparatus in accordance with the present invention, a system for promoting a more uniform distribution of radial and axial stresses (e.g., forces over an area) and deflections along soft tissue may minimize the potential for trauma and damage to soft tissue during soft tissue exercise. Trauma may be manifest in the form of pain, bruising, and discoloration. Damage may be structural, such as deformity or broken capillaries, or functional, such as ejaculation impairment.

Apparatus and methods for promoting the expansion of soft tissue have been found to minimize local trauma and promote soft tissue that is healthier and has improved vascular function as well as an overall permanent increase in axial and radial size while in flaccid and erect conditions. Additionally, the promotion of healthier soft tissue appears to result in decreased dependence on a vacuum device for initiation and maintenance of function, such as an erection. This may further result in reducing the potential impairment to ejaculation.

In this manner, referring to FIG. 1 a process flow diagram illustrates a method 10 for exercising a member formed of soft tissue using one embodiment of an apparatus and method in accordance with the present invention. The method 10 may include the steps of providing 12 a soft-tissue expansion apparatus, deploying 14 a soft-tissue expansion apparatus, and conducting 16 soft-tissue expansion exercise.

Providing a soft-tissue expansion apparatus 12 may include providing a chamber having a wall of fixed dimension, providing an evacuation device to reduce ambient pressure in the chamber, and providing a membrane, substantially tubular in shape, substantially elastic, having an interior passage, having a first end sealing securable to the wall of the chamber, and having a second end extendable into the chamber a length greater than the length of a member comprising soft tissue.

Deploying 14 a soft tissue expansion apparatus may include occluding the interior passage of the membrane by the member, in a flaccid state. Conducting 16 a soft tissue expansion exercise may include operating the evacuation device to reduce the ambient pressure in the chamber, drawing the member into the interior passage by virtue of a pressure differential between bodily vascular pressure and the ambient pressure, and expanding the member axially and radially due to the pressure differential.

A pressure differential may be maintained at a value and for a time selected to promote vascular expansion and expansion of a soft tissue member against a bias provided by the elasticity of the membrane. A pressure differential in the chamber may be released by opening the chamber to atmospheric pressure. Releasing the pressure differential will promote vascular contraction and the flow of blood from the soft tissue member back into the body. Alternatingly repeating both the operating of the evacuation device and releasing the pressure differential a number of times promotes vascular flexibility within the soft tissue member in axial and radial directions. Alternatingly repeating the operating of the evacuation device and releasing the pressure differential a number of times also promotes permanent expansion in the member.

Vacuum devices for promoting expansion of soft tissue may lead to significant trauma, due to rupture of cellular capillaries and manifest in the form of pain, bruising and discoloration. Such trauma can result from contact pressure between apparatus hardware and soft tissue, proximity of local blood vessels to skin surface, and reduction in muscular tone on or nearby the soft tissue.

This trauma may be localized to the area where a vacuum device is being employed. In addition, soft tissue trauma may also extend to the abdominal wall or other soft tissue outside of the localized area. Still further, use of vacuum devices may lead to functional damage, such as impairment of ejaculation. The method 10 may help to reduce soft tissue trauma while improving the efficiency of soft tisssue exercise and overall may lead to a healthier soft tissue area.

Certain embodiments of an apparatus and method in accordance with the invention can provide support to soft tissue undergoing reduced pressure enlargement. This support helps to minimize local weaknesses in the soft tissue that allow for trauma and damage due to the unequal response (e.g., deflection) resulting from application of decreased external pressure. More specifically, unequal application or distribution of pressure to soft tissue in a reduced pressure environment results in ballooning or swelling in local blood vessels or capillaries.

Ballooning or swelling of blood vessels, if left unapposed, results in an aneurysm or microaneurysm and disruption or damage to the blood vessel wall. This disruption or damage to the blood vessel wall allows for the extravascular or subdermal accumulation of blood and other tissues and may lead to swelling, bruising, deformity, discoloration, and other soft tissue trauma. A system to minimize ballooning or swelling of blood vessels in soft tissue undergoing enlargement under reduced pressure meets the need for a safer and more efficient soft tissue exercise conditioning program.

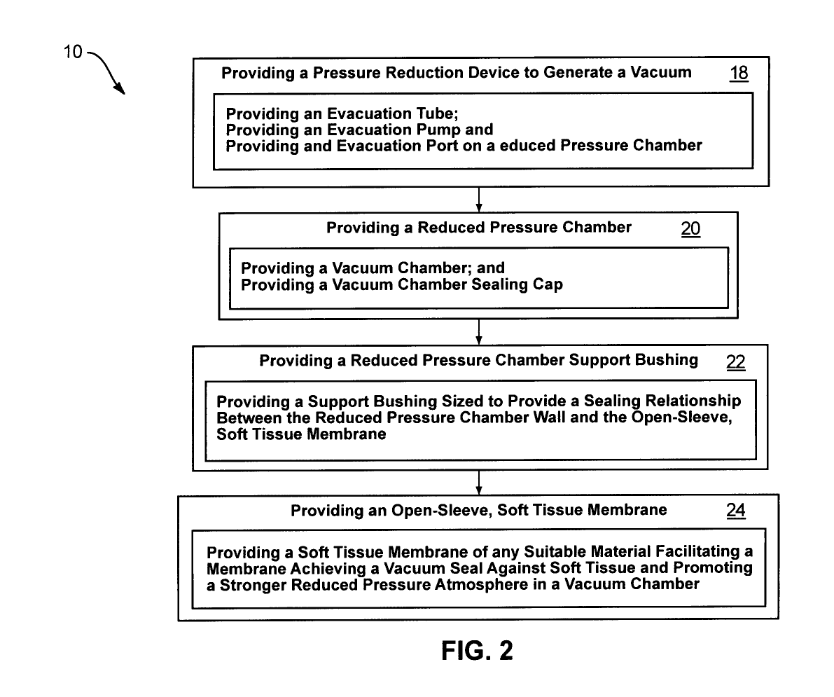

Referring to FIG. 2 a process flow diagram illustrates providing a soft-tissue expansion apparatus 12 of the method 10 of FIG. 1 in one embodiment of an apparatus and method in accordance with the present invention. FIG. 2 illustrates a soft tissue expansion apparatus 12 further including providing 18 a vacuum source, providing 20 a vacuum chamber, providing 22 a bushing, and providing 24 a membrane. Any suitable vacuum source 18 and vacuum chamber 20 may be utilized in the practice of the present invention. However, providing 18 a vacuum source and providing 20 a vacuum chamber in combination with providing 22 a bushing and providing 24 a membrane, have not previously been proposed.

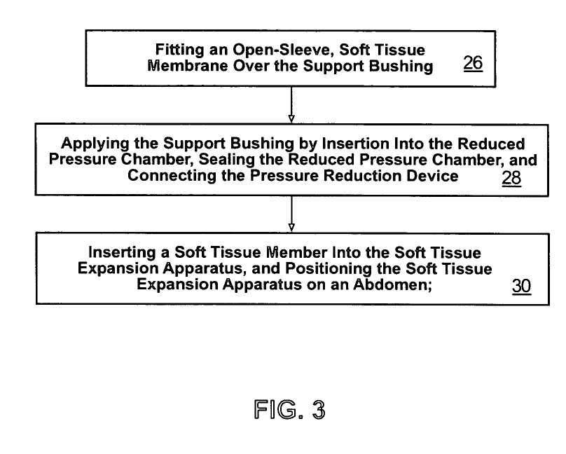

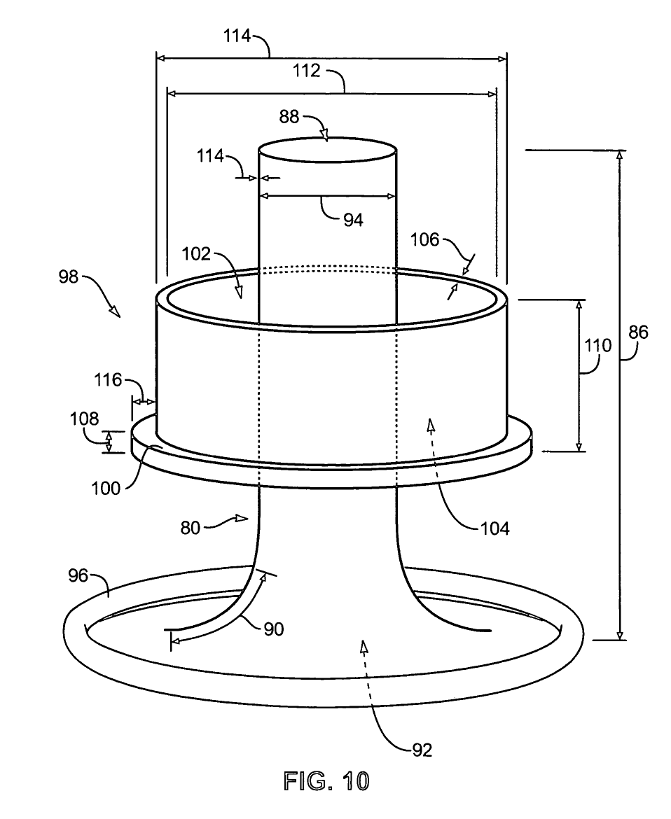

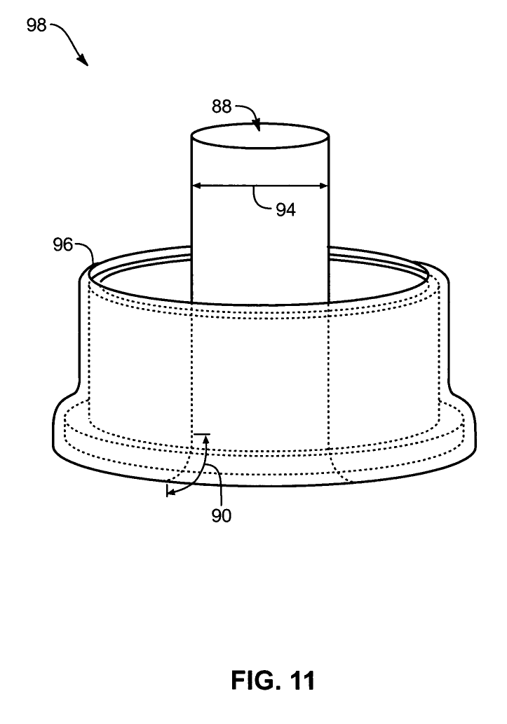

Referring to FIG. 3 a process flow diagram illustrates deploying 14 a soft-tissue expansion apparatus of the method 10 of FIG. 1 in one embodiment of an apparatus and method in accordance with the present invention. Deploying 14 an apparatus may further include fitting 26, application 28, and insertion 30. Fitting 26 may include the fitting of a bushing 98 over the outer surface of a membrane 80 as illustrated and described in FIGS. 10 and 11. Application 28 may include assembling the vacuum source 18 and vacuum chamber 20 and placement of the vacuum chamber over a membrane 118 conformed to fit over the bushing. Application 28 may also include the introduction of a gel sealant 128 to the inner surface 129 of a membrane 80. A gel sealant 128 may serve to promote a uniform distribution of radial 140 and axial 142 stresses to soft tissue both by transferring pressure and reducing surface friction opposing relative motion. In addition, a gel sealant 128 may likewise promote a reduction in trauma to an abdominal wall 126 or surrounding soft tissue. Insertion 30 may include the introduction of a flaccid member 120 into the membrane afferent aperture 92.

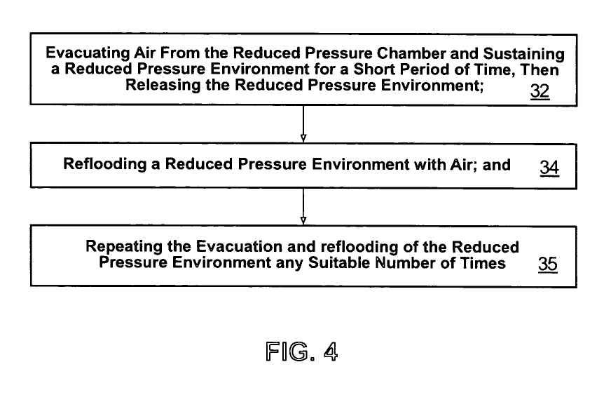

Referring to FIG. 4 a process flow diagram illustrates conducting 16 a soft-tissue expansion exercise of the method 10 of FIG. 1 in one embodiment of an apparatus and method in accordance with the present invention. Conducting 16 a soft-tissue expansion exercise may be analogous to conducting a soft-tissue exercise conditioning program. Conducting 16 a soft-tissue expansion exercise may further include cyclic evacuation 32 and reflood 34. Evacuation 32 may utilize a vacuum source 18 to evacuate air from a vacuum chamber 50.

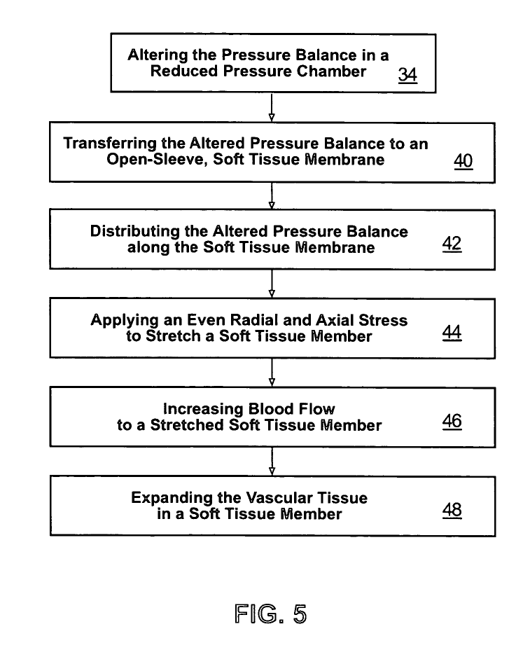

Evacuation 32 of air from a vacuum chamber 50 may promote the expansion of soft tissue as illustrated in FIGS. 5, 12 and 13 and associated text. Once the desired expansion of soft tissue has been achieved with a vacuum device, a resulting soft tissue expansion may be maintained for a short period of time by maintaining the vacuum chamber 50 about the soft tissue. Reflood 34 of ambient air into the chamber 50 will equalize the pressure in the vacuum chamber 50 with the ambient pressure in the atmosphere surrounding the vacuum chamber 50. Reflood 34 may lead to a decrease in soft tissue size. Evacuation 32 and reflood 34 may be repeated 35 any suitable number of times to conduct 16 a soft tissue exercise 16 and a soft tissue exercise conditioning program.

Referring to FIG. 5 a process flow diagram illustrates a method 10 for promoting expansion of soft tissue using in one embodiment of an apparatus and method in accordance with the present invention. A method 10 for promoting expansion of soft tissue may further include alteration of a pressure balance 38, pressure transduction 40 to membrane, pressure distribution 42, application of radial and axial stresses 44, increased blood flow 46, and vascular expansion 48. Alternation of a pressure balance 38 may occur upon evacuation 32 of air from a vacuum chamber 50. Any suitable apparatus for evacuation 32 of air may be utilized.

As previously discussed, application of local pressure by forces and devices, as well as the unequal response of the body to application of reduced pressures to unsupported soft tissue often results in blood vessel damage, such as aneurysm, microaneurysm, and disruption of vessel walls. Upon alteration of a pressure balance 38, a cascade of events may occur. This cascade may include transduction of pressure to a membrane 80. A membrane 80 may distribute the alteration in pressure leading to a uniform application of radial 140 and axial 142 stresses along an area of soft tissue. Stated in another way, a membrane 80, may have an elastic property urging all tissue thereunder to move in compliance therewith, and urging it to return to a normal condition following expansive deformation.

Elastic properties are typically described under the physical law called Hooke's law. An object may be said to exhibit Hooke's law if the object moves under the influence of a restoring force and may be expressed by the formula F=(−k)(x), where F is a restoring force, k is a force constant, and x is the displacement of an object from its equilibrium position.

In objects with elastic properties, the force constant, k, is commonly referred to as a coefficient or constant of elasticity and also sometimes referred to as a coefficient of resistance. The coefficient of resistance is the quotient of a stress (of a given kind), by the strain (of a given kind) which it produces. Bias is used to describe the amount of displacement from an equilibrium position that may occur to an object. In certain embodiments of an apparatus and method in accordance with the invention, damage to soft tissue, as a result of a reduced pressure environment is minimized by the biasing (pressure and deflection) due to the elastic properties of a membrane 80.

When radial 140 and axial 142 stresses are applied to a flaccid member 120 there may be an increase in blood flow, such as along the penile, urethral, cavemosa and dorsal arteries. This increase in blood flow may result in vascular expansion 48. As vascular expansion 48 occurs in a reduced pressure environment, there may be an increase of aneurysm or microaneurysm in unsupported soft tissue. A membrane 80 functions as a supportive sleeve to regulate the application of pressure to a soft tissue member.

Based on the foregoing, it will be readily apparent that a variety of methods for promoting expansion of soft tissue may be performed in accordance with the inventive principles set forth herein. It is intended, therefore, that the examples provided herein be viewed as exemplary of the principles of the present invention, and not as restrictive to a particular structure, method, step, or ordering of steps for implementing those principles.

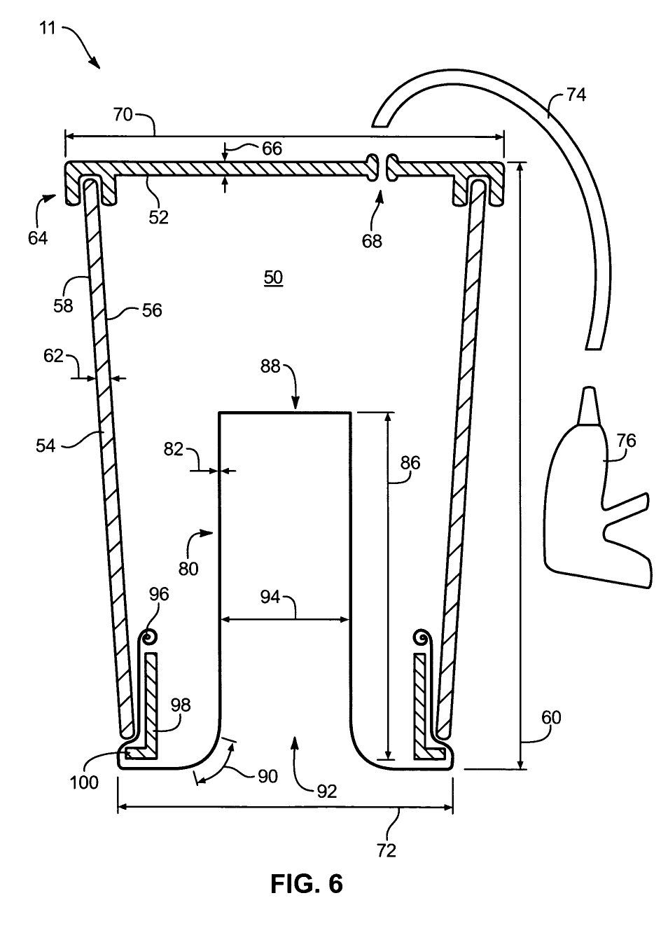

Referring to FIG. 6, one embodiment of an apparatus 11 for promoting expansion of soft tissue is illustrated. The apparatus 11 for expansion of soft tissue may include a vacuum chamber 50 and a vacuum chamber sealing cap 52 that fits onto the vacuum chamber efferent aperture width 70 using a connection 64, such as by a tongue and groove configuration or other suitable, sealing shape. The apparatus 11, may also include an evacuation tube 74, an evacuation pump 76, a membrane 80 and a bushing 98.

A vacuum chamber 50 may have a vacuum chamber wall 54, which is generally substantially cylindrical in shape. A vacuum chamber wall 54 may have a vacuum chamber wall inner surface 56 and a vacuum chamber wall outer surface 58. A vacuum chamber wall thickness 62 may be sufficient to withstand the appropriate pressure difference being generated in a vacuum chamber 50. A vacuum chamber sealing cap sealing thickness 66 also should be sufficient to sustain the appropriate pressure difference being generated in a vacuum chamber 50. A vacuum chamber height 60 may be in the range of about six (6) to twelve (12) inches. Moreover, a vacuum chamber afferent aperture width 72 may be in the range of about two and one-half (2.5) to three and one-half (3.5) inches. A vacuum chamber efferent aperture width 70 may be in the range of about three and one-half (3.5) inches to four and one-half (4.5) inches. One embodiment of the vacuum chamber wall 54 may be in the general form of a taper or frustum of a cone. The narrower end coincides with the vacuum chamber afferent aperture 72 and the wider end coincides with the vacuum chamber efferent aperture 70.

A vacuum chamber sealing cap evacuation port 68 may be used in connection with an evacuation tube 74 and an evacuation pump 76 to evacuate 32 air from a vacuum chamber 50. This evacuation 32 of air may create a reduced pressure atmosphere within a vacuum chamber 50.

A membrane 80, when deployed, particularly, may further include a membrane neck 84, a membrane height 86, a membrane thickness 82, a membrane tapered base 90 and a membrane rolled flange 96. Additionally, a bushing 98 may include a bushing lip 100.

Based on the foregoing, a variety of apparatus for promoting expansion of soft tissue may be created in accordance with the inventive principles set forth herein. It is intended, therefore, that the illustrations provided herein be viewed as exemplary of the principles of the present invention, and not as restrictive to a particular structure, method, step, or ordering of steps for implementing those principles.

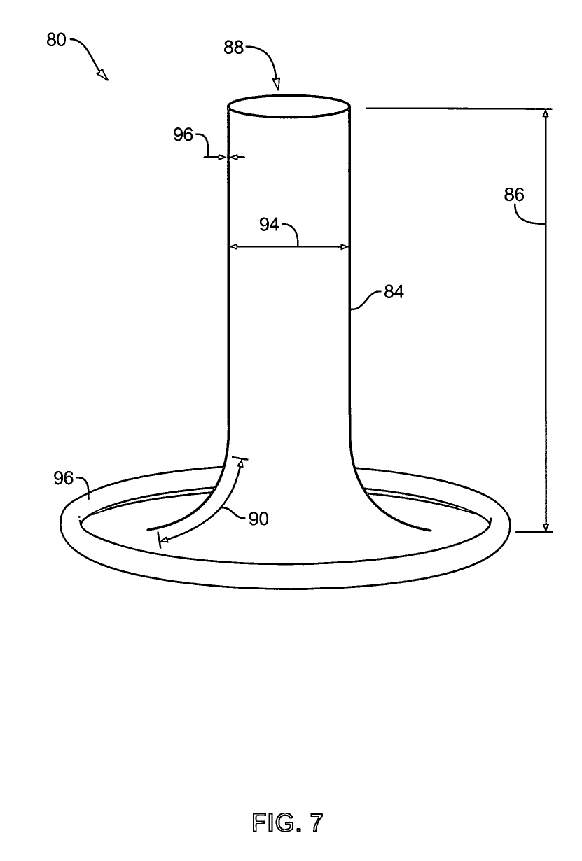

Referring to FIG. 7 a side, perspective view provides more detail of a membrane 80 used in an apparatus for providing a more even distribution of axial 142 and radial 140 stresses and deflections applied to soft tissue. A membrane 80 may further include a membrane thickness 82 in the range of from about two thousandths (0.002) to about one thirty-second (1/32) inch. Very soft or flexible materials may even have a membrane thickness 82 up to one-eighth (⅛) inch.

A membrane diameter 94, unstretched, may be in the range of from about three-fourths (¾) to about one and one-half (1.5) inches, and typically one (1) inch. It may have a value of about up to three (3) inches when stretched. A tapered base 90 may form an afferent aperture 92 through a gradual diminution in membrane diameter. A membrane height 86 may be in the range of from about (4) to about nine (9) inches depending on an exercise regimen. A membrane 80 may also have an afferent aperture 92 and an efferent aperture 88 which may form, or define a hollow, elastic, cylindrical tube.

A membrane 80 may have a rolled flange 96, and may be formed of any suitable material facilitating its functions. A membrane 80 achieves a vacuum seal against soft tissue and promotes a stronger reduced pressure atmosphere within a vacuum chamber 50. The sealing material (e.g., gel) may also provide lubrication for relative axial motion between the membrane and soft tissue. A membrane 80 may be conformed to fit over the exterior surface of a bushing 98 and provides an inward bias pressure against a member.

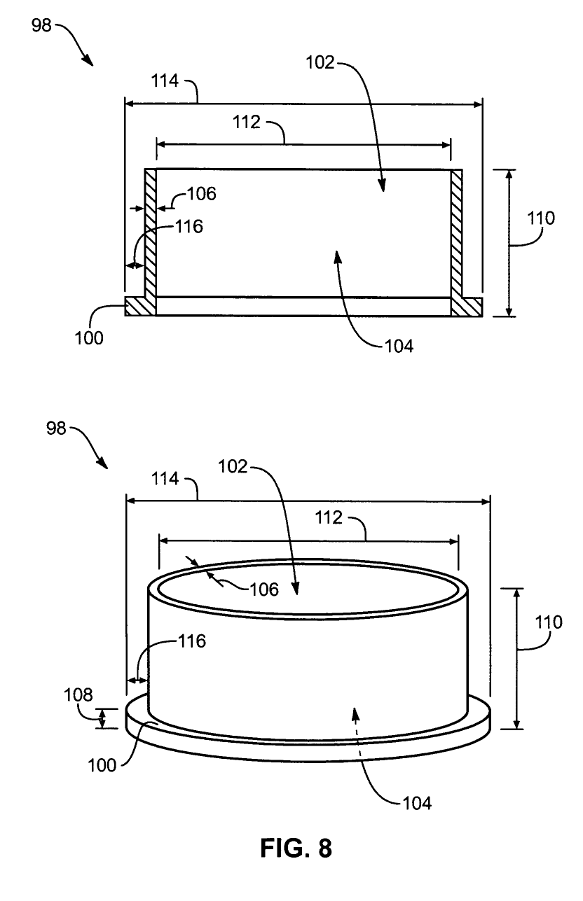

Referring to FIG. 8 a side, perspective view provides more detail of a support bushing used in one embodiment of an apparatus in accordance with the present invention. A bushing 98 may be used to provide a base for resting a vacuum chamber 50 or other reduced pressure environment apparatus. A bushing 98 is fitted to the chamber and sized to hold a membrane 80 against the chamber wall 54 in a sealing relationship. A bushing 98 may be positioned inside the chamber or outside the chamber wall with respect to the interior of the chamber. A bushing 98 may also be configured in a manner to allow at least partial insertion into a vacuum chamber afferent aperture width 72.

In certain embodiments of an apparatus in accordance with the invention, a bushing 98, a membrane 80, and a vacuum chamber wall 54 may be placed in a sealing relationship. One example is as sandwich configuration, wherein a membrane 80 is between a vacuum chamber wall 54 and a bushing 98, to promote an airtight seal within a vacuum chamber 50.

A bushing 98 may have an approximately cylindrical shape with an efferent (exit) aperture 102, an afferent (entrance) aperture 104 and a bushing lip 100. A bushing 98 may have a wall height 110 in the range of from about one-half (½) inch to about three (3) inches and may have a wall thickness 106 in the range of from about one-sixteenth (1/16) inch to about one-half (½) inch. In one embodiment, the height 110 is one (1) inch and the wall thickness 106 is one-fourth (¼) inch. A bushing 98 may have an inner diameter 112 in the range of from about one and one-half (1.5) inches to about three (3) inches.

A bushing outer diameter 114 may be in the range of from about one and three-fourth (1.75) inches to about five (5) inches. The difference in width between a bushing inner diameter 112 and outer diameter 114 is primarily determined by a lip width 116. A bushing lip 100 may also have a lip height 108 in the range of from about one-eighth (⅛) inch to about one-half (½) inch. Additionally, a bushing lip 100 may provide an efficient vacuum seal by serving as a surface rest for the bottom edge of a vacuum chamber wall 54.

In an alternative embodiment of an apparatus in accordance with the present invention, a bushing lip 100 may include a recessed channel for enhancing the surface area contact between a vacuum chamber wall 54, a membrane 80 and a support bushing lip 100. A recessed channel may have a curved, square, or polyhedral base. A recessed channel may be configured to conform to the bottom edge formed by a vacuum chamber wall 54.

In certain embodiments of an apparatus and method in accordance with the invention, a plurality of additional sealing relationships between a membrane 80, bushing 98, and vacuum chamber wall 54 are contemplated. For example, but not limiting of the invention, a sealing relationship between a vacuum chamber wall 54, membrane 80, and bushing 98 may be configured wherein a membrane 80 is threaded between a bushing 98 inner surface and the outer surface of a vacuum chamber support wall 54. A sealing relationship may also be configured wherein a membrane 80 is threaded between a bushing 98 outer surface and the inner surface of a vacuum chamber support wall 54.

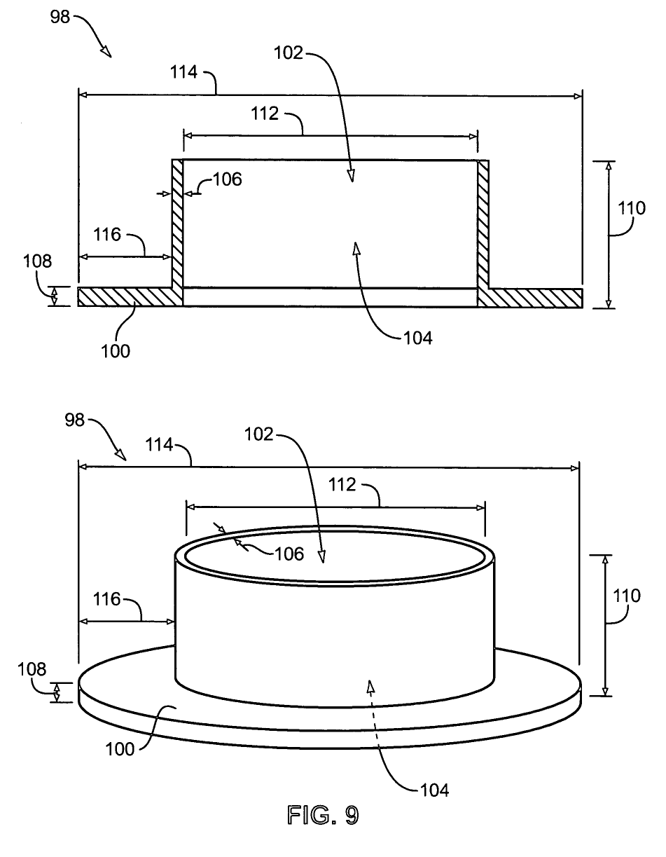

Referring to FIG. 9 a side, perspective view provides more detail of a bushing 98 used in a system for providing a more even distribution of axial and radial stresses and deflections along soft tissue in an alternative embodiment of an apparatus in accordance with the present invention. In this alternative embodiment, a lip width 116 may extend outwardly from the center of a bushing 98 creating a larger bushing lip 100. In addition to providing an efficient vacuum seal, as described in FIG. 8, a larger bushing support 100 may also provide more support for an abdominal wall 126 or other soft tissue area which is a direct target of apparatus 11. A larger bushing lip 100 may result in less trauma, such as pain, bruising and discoloration, to soft tissue during a soft tissue exercise.

Based on the foregoing, it will be readily apparent that a variety of apparatus for a bushing 98 may be performed in accordance with the inventive principles set forth herein. It is intended, therefore, that the examples provided herein be viewed as exemplary of the principles of the present invention, and not as restrictive to a particular structure, step, or ordering of steps for implementing those principles.

Referring to FIG. 10, more detail is provided regarding fitting a bushing 98 over a membrane 80 in one embodiment of an apparatus and method in accordance with the present invention. A bushing 98 and a membrane 80 may be positioned so that the membrane efferent diameter 88 and membrane neck 84 may be inserted through a bushing 98 at a bushing afferent diameter 104. A bushing may be fitted over the top surface of a membrane until a bushing lip 100 is in contact with a membrane 80 at a location between a membrane tapered neck 90 and a membrane rolled flange 96.

Referring to FIG. 11, a membrane 80 conforms to fit over a bushing 98. Following the initial fitting described in FIG. 10 above, a membrane 80 may conform to fit over the exterior surface of a bushing 98. A membrane 80 may be stretched along the plane section between a membrane tapered base 90 and a rolled flange 96 to fit over the bushing efferent aperture 102.

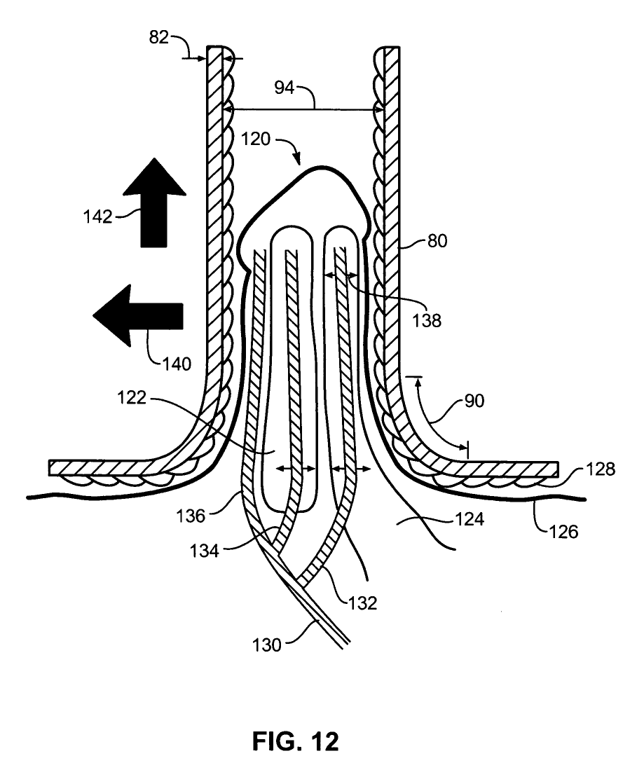

Referring to FIG. 12, a physiological illustration of the method illustrated in FIG. 5 provides more detail regarding a membrane 80 used in a system for providing an even distribution of axial 142 and radial 140 stresses along flaccid soft tissue 120 in one embodiment of an apparatus and method in accordance with the present invention. A soft tissue vacuum exercise device may work more efficiently if a better seal is achieved in the vacuum chamber area surrounding the soft tissue. Such a seal may be achieved using an apparatus 11.

In addition to achieving a better seal, a membrane 80 having a membrane thickness 82 in the range of from about two thousandths (0.002) to about one thirty-second (1/32) inch (very soft or flexible materials could have a membrane thickness 82 up to one eighth (⅛) inch). A membrane diameter 94, unstretched, may be in the range of from about three-fourths (¾) to about one and one-half (1.5) inches, and typically one (1) inch. It may have a value of about up to three (3) inches when stretched. A tapered base 90 may form an afferent aperture 92 through a gradual diminution in membrane diameter. A membrane height 86 may be in the range of from about four (4) to about nine (9) inches depending on an exercise regiment. A membrane 80 may also have an afferent aperture 92 and an efferent aperture 88 which may form, or define a hollow, elastic, cylindrical tube.

A sealant 128, such as a gel, may be applied to a membrane 80 prior to inserting a flaccid member 120. A sealant gel 128 may be used to create a better (e.g., air-tight) seal between a flaccid member 120, and between an abdominal wall 126, and a membrane 80. This seal may enhance the pressure distribution 42, relative motion, and axial pressure draw, and may result in a uniform application 44 of radial 140 and axial 142 stresses along soft tissue.

Evacuation 32 of air from a vacuum chamber 50 may alter the pressure balance 38 and create a reduced pressure atmosphere around soft tissue. This reduced pressure atmosphere may be transduced 40 to a membrane 80. A membrane 80 may undergo pressure distribution 42 and may lead to a uniform application of radial 140 and axial 142 stresses along a flaccid member 120 or other soft tissue and will contain the member against localized deflections.

Application of radial 140 and axial 142 stresses has been observed to result in stretching soft tissue. Soft tissue stretching has been observed to result in increased blood flow 46 to a flaccid member 120 and also to result in vascular expansion 48 and health. Within a flaccid member 120 or an erect member 144, there are two (2) types of fibrous cylindrical tubes, the corpus cavernosa 122 and the corpus spongiosum 124. Typically, there are two corpus cavernosa 122 (“corpora cavemosa”) and a single corpus spongiosum 124. The corpus spongiosum 124 encloses the urethra and also encloses a urethral artery 132. Each corpus cavernosa 122 encloses a cavernosa artery 134 and has a dorsal artery 136, which runs along the dorsal surface.

Vascular expansion 138 results from an alteration of the pressure balance 38 within a vacuum chamber 50. Alternation of the pressure balance is transduced 40 to a membrane 80 and is dispersed 42 under the application 44 of radial 140 and axial 142 stresses. An increased blood flow 46 to soft tissue may occur through a penile artery 130, urethral artery 132, cavemosa artery 134, and dorsal artery 136. An increased blood flow 46 into an area subjected to a reduced pressure atmosphere (i.e., a vacuum) may promote the expansion of a flaccid member 120.

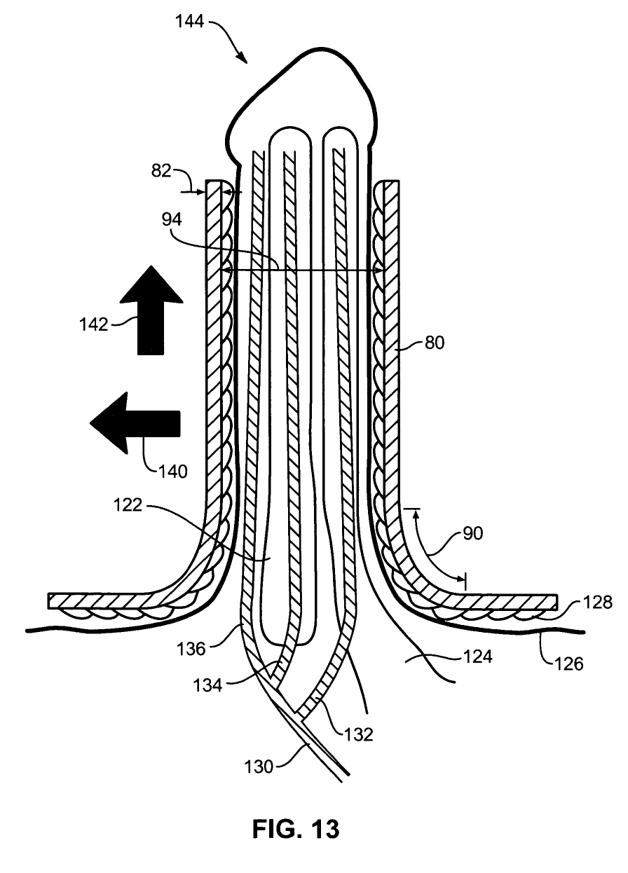

Referring to FIG. 13, more detail is provided regarding FIG. 12, a physiological illustration of the method illustrated in FIG. 5, in one embodiment of an apparatus and method in accordance with the present invention. Following an increased blood flow 46 to soft tissue occurring through a penile artery 130, urethral artery 132, cavemosa artery 134, and dorsal artery 136, an increased blood flow 46 and reduced ambient pressure promotes expansion of an erect member 144 extending through a membrane efferent aperture 88 toward and within a vacuum chamber 50.

A sealant gel 128 may be applied to a membrane 80 prior to inserting a flaccid member 120. A sealant gel 128 may be used to create an airtight seal between a flaccid member 120 (and abdominal wall 126) and a membrane 80. This seal enhances the pressure distribution 42 and results in a uniform application 44 of radial 140 and axial 142 stresses along soft tissue. Meanwhile, the membrane 80 resists localized anomalies that might cause trauma or local distension. A uniform application 44 of radial 140 and axial 142 stresses along soft tissue, and restraint against localized deflection due to over compliance, results in less trauma to soft tissue from an apparatus and method for promoting soft tissue expansion with a vacuum exercise device.

Since the apparatus 11 and methods for soft-tissue expansion are configured to apply to any number of embodiments for practicing the present invention. It will be readily appreciated that changes may be made to this example without departing from the invention.

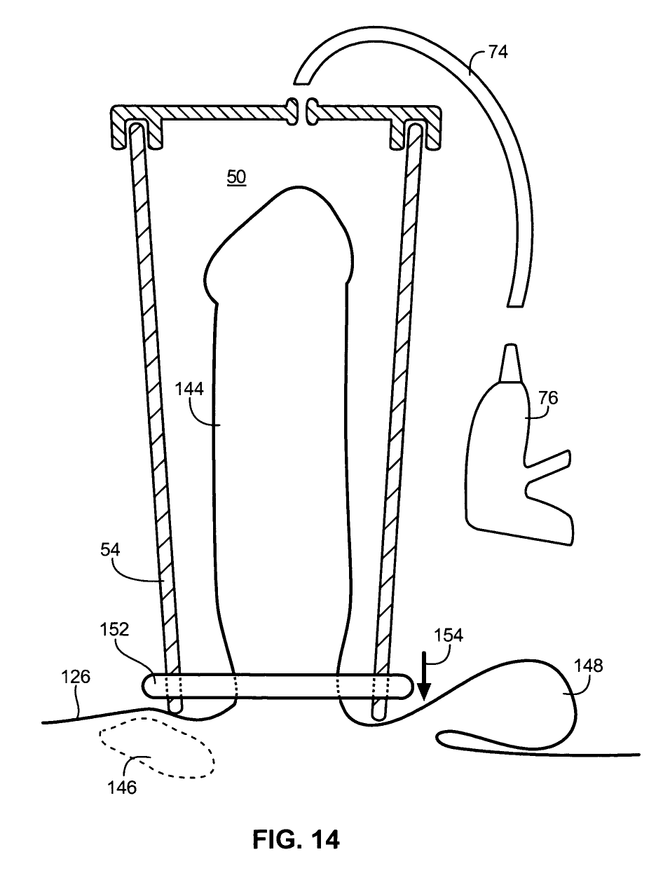

Referring to FIG. 14, an embodiment of an apparatus and method in accordance with the present invention is depicted in which one or more restriction bands 152 are used to minimize early detumescence or to maximize the rigidity of a vacuum generated erection upon removal of the vacuum source. One or more restriction bands 152, typically in the form of an elastic or otherwise flexible clamp, may be placed around the outside diameter of a vacuum chamber wall 54 at the afferent end holding the bands in an open (e.g., extended, stretched, large diameter) position.

A flaccid member 120 is introduced into the afferent end of the vacuum chamber 50 until the chamber comes to rest against the abdominal wall 126. The superior side of the vacuum chamber 50 is placed against the abdominal wall 126 in a position just below the public bone 146 and just above the scrotum 148. As a reduced pressure environment (e.g., vacuum) is being generated in the vaccum chamber 50 and the soft tissue member begins to expand, the abdominal wall tends to be compressed and the base of the penis, sometimes called the oz 150, is formed into a conical shape. The frustum is this conical shape is directed in a posterior direction.

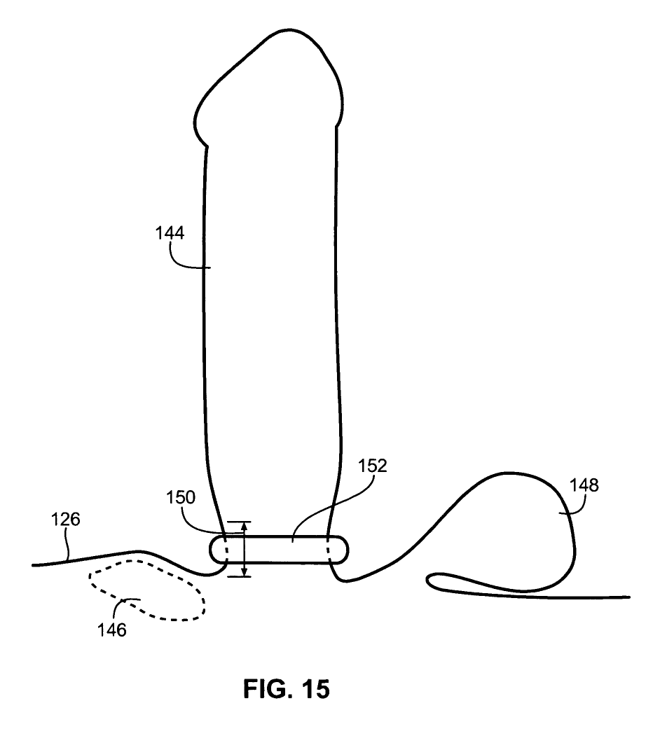

Referring now to FIG. 15, upon reaching a desired level of soft tissue expansion, one or more restricting bands 152 are moved 154 over the afferent end of the vacuum chamber 50 and placed into contact with the erect member 144 at about the oz 150. The oz 150 is outwardly sloped and closely conforms with one or more tensile tissues which promote an erect member. Placement of one or more restriction bands at the oz 150 may further compress the abdominal wall against one or more tensile tissues and facilitate the strength and/or duration of a member in an erect state.

As the blood flow to a flaccid member 120 is increased and as erectile tissues are stretched and tightened, the abdominal wall may be compressed to facilitate the oz 150 being formed into a conical shape. Therefore, one or more restriction bands 152 placed at the oz 150 may minimize too early detumescence of an erection or maximize rigidity of a vacuum generated erection, upon removal of a vacuum source, for a period of time.

The present invention may be embodied in other specific forms without departing from its essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

1. An apparatus for promoting radial and axial expansion of soft tissue, the apparatus comprising:
a chamber having a wall defining an interior thereof and supporting an ambient pressure therein;
a vacuum source connected to the chamber to reduce the ambient pressure therein; and
a membrane sealingly connected to the chamber and characterized by a constant of elasticity selected to provide a bias resisting the reduction of ambient pressure against a soft tissue member in the membrane in order to reduce localized trauma to the soft tissue due to the reduction of ambient pressure.

2. The apparatus of claim 1 wherein the membrane is substantially cylindrical in shape.

3. The apparatus of claim 2, wherein the membrane has a proximate end sealed against the wall of the chamber and a distal end open to the interior thereof.

4. The apparatus of claim 3, wherein the membrane is selected to promote more even distribution of axial and radial stress against soft tissue held therewithin.

5. The apparatus of claim 1, further comprising a bushing fitted to the chamber and sized to hold the membrane against the chamber in sealing relation thereto.

6. The apparatus of claim 5, wherein the bushing is positioned inside the chamber.

7. The apparatus of claim 5, wherein the bushing is positioned outside the chamber wall with respect to the interior of the chamber.

8. The apparatus of claim 5, wherein the bushing further comprises:
a cylindrical portion extending into the chamber
an afferent aperture portion extending between the wall of the chamber and the cylindrical portion to receive soft tissue thereinto; and
an afferent aperture portion open to the chamber to permit exit of the soft tissue thereinto under the influence of the reduced ambient pressure.

9. The apparatus of 5, wherein the bushing is made from a material selected from the group consisting of an elastomeric material, a plastic material, a polymer, a reinforced polymer, an expanded polymer, and a metal.

10. The apparatus of claim 5, wherein the bushing further comprises a lip form to preclude insertion of the bushing completely into the chamber.

11. The apparatus of claim 10, wherein the lip is sized to form an abdominal seal and abdominal wall support for a user.

12. The apparatus of claim 1, wherein the membrane has a first end and second end and further comprises:
a rolled flange proximate the first end;
a cylindrical portion defining an efferent aperture proximate the second end; and
a tapered portion defining an afferent aperture between the first and second ends.

13. A method for exercising a member formed of soft tissue, the method comprising:
providing a chamber having a wall of fixed dimension;
providing an evacuation device to reduce ambient pressure in the chamber;
providing a membrane, substantially tubular in shape, substantially elastic, having an interior passage, having a first end sealing securable to the wall of the chamber, and having a second end extendable into the chamber a length greater than the length of a member comprising soft tissue;
occluding the interior passage of the membrane by the member, in a flaccid state;
operating the evacuation device to reducing the ambient pressure in the chamber;
drawing the member into the interior passage by virtue of a pressure differential between bodily vascular pressure and the ambient pressure; and
expanding the member axially and radially due to the pressure differential.

14. The method of claim 13, further comprising maintaining the pressure differential at a value and for a time selected to promote vascular expansion and expansion of the member against a bias provided by the elasticity of the membrane.

15. The method of claim 13, further comprising opening the chamber to atmospheric pressure to release the pressure differential.

16. The method of claim 13, further comprising releasing the pressure differential to promote vascular contraction and flow of blood from the member back into the body.

17. The method of claim 16, further comprising alternatingly repeating both the operating the evacuation device and releasing the pressure differential a number of times selected to promote permanent vascular flexibility within the member in axial and radial directions.

18. The method of claim 16, further comprising alternatingly repeating the operating the evacuation device and releasing the pressure differential a number of times selected to promote permanent vascular expansion in the member.

19. The method of claim 16, further comprising alternatingly repeating operating the evacuation device and releasing the pressure differential, while radially modifying pressure on the member by the elastic bias of the membrane to minimize localized tissue damage thereto, a number of times selected to promote permanent expansion of the member.

20. The method of claim 19, further comprising applying a sealant between the membrane and the member to promote sealing therebetween and reduce friction to promote relative axial motion therebetween.

21. The method of claim 19, further comprising applying a sealing gel to the membrane for sealing the member and abdominal wall of a user there against to enhance the pressure differential applied thereto by the membrane.
